(19) United States
(12) Patent Application Publication
(10) Pub. No.: US 2008/0280290 A1
(43) Pub. Date: Nov. 13, 2008

(54) Method and Device for the Collection and Isolation of Nucleic Acid

(76) Inventors: Elliott P. Dawson, Murfreesboro, TN (US); Kristie E. Womble, Franklin, TN (US)

Correspondence Address: SHELDON MAK ROSE & ANDERSON PC, 100 Corson Street, Third Floor, PASADENA, CA, 91103-3842, US

(21) Appl. No.: 10/599,248

(22) PCT Filed: Aug. 18, 2006
(86) PCT No.: PCT/US2006/032531

§ 371 Date: Sep. 22, 2006

Related U.S. Application Data
(60) Provisional application No. 60/709,869, filed on Aug. 19, 2005.

(51) Int. Cl. C12Q 1/68; C12M 3/00

(52) U.S. Cl. 435/6; 435/287.2

(57) ABSTRACT

A device for collecting and preserving nucleic acids in a sample, the device comprising: a) a support; b) one or more than one sample zone in the support for loading the sample onto the device; and c) a composition comprising i) one or more than one absorbent, and ii) one or more than one stabilizer; where the one or more than one sample zone on the support comprises a recess or space within the support extending from the top surface toward, but not through, the bottom surface, or comprises a space within the support and the composition is retained within the sample zone. A method for collecting and preserving nucleic acids in a sample, the method comprising a) providing a device for collecting and preserving nucleic acids in a sample according to the present invention; b) providing a sample potentially comprising one or more than one nucleic acid; and c) applying part or all of the sample to one or more than one of the sample zones on the device. A method of detecting and quantifying nucleic acids in a sample, the method comprising a) collecting and preserving nucleic acids in the sample according to a method of the present invention; b) removing the absorbent with sample from the sample zones of the device; and c) detecting, or detecting and quantifying the nucleic acids.

### CROSS REFERENCE TO RELATED APPLICATIONS

The present application claims the benefit of U.S. Provisional Patent Application 60/709,869, titled “A Method for the Collection and Isolation of mRNA,” filed Aug. 19, 2005; the contents of which are incorporated in this disclosure by reference in their entirety.

### BACKGROUND

Minute amounts of mRNA can be detected and quantified from a few cells using highly sensitive, gene specific methods, such as for example qRT-PCR combined with general methods for the amplification of mRNA. mRNA to be detected and quantified is often isolated from whole blood for many reasons, including in studies of human disease. Disadvantageously, however, special collection techniques, resources and supplies are required for collecting the relatively large volumes of whole blood needed for isolating mRNA from the whole blood, before the mRNA is detected and quantified. For example, presently available RNA blood collection tubes can only be used with certain isolation methods, permit storage of the sample for a very limited time at room temperature, and sometimes alter the quality of RNA obtained. Further, presently available RNA blood collection tubes can be inconvenient to use in large multi-subject studies.

Therefore, there remains a need of a device for collecting and preserving nucleic acids in a sample that are not associated with the disadvantages of presently available RNA blood collection tubes. Further, there remains a need of a method for collecting and preserving nucleic acids in a biological sample that are not associated with these disadvantages.

### SUMMARY

According to one embodiment of the present invention, there is provided a device for collecting and preserving nucleic acids in a sample. The device comprises: a) a support comprising a top surface, an opposing bottom surface, and a lateral edge surrounding the top surface and the bottom surface; b) one or more than one sample zone in the support for loading the sample onto the device; and c) a composition comprising i) one or more than one absorbent, and ii) one or more than one stabilizer; where the one or more than one sample zone on the support comprises a recess or space within the support extending from the top surface toward, but not through, the bottom surface, or comprises a space within the support extending from the top surface completely through the bottom surface, and where the composition is retained within the sample zone. In one embodiment, the support comprises a hydrophobic material. In another embodiment, the support comprises a material selected from the group consisting of plasticized cardboard, polyacetate, polycarbonate and polypropylene. In another embodiment, the device further comprises a shape selected from the group consisting of an oval, a circle, a rectangle, a rectangle with rounded corners, a square, a square with rounded corners, a triangle and a triangle with rounded corners. In another embodiment, the one or more than one sample zone comprises a plurality of sample zones comprising between 2 and 1000 sample zones. In another embodiment, the one or more than one sample zone comprises a plurality of sample zones comprising between 2 and 500 sample zones. In another embodiment, the one or more than one sample zone comprises a plurality of sample zones comprising between 20 and 200 sample zones. In another embodiment, the device comprises a plurality of sample zones, and the shape of each sample zone is identical to every other sample zone. In another embodiment, the device comprises a plurality of sample zones, and the shape of at least one sample zone is different that the shape of at least one other sample zone. In another embodiment, the shape of at least one of the one or more than one sample zone, as viewed from the top surface, comprises a shape selected from the group consisting of an oval, a circle, a rectangle, a square and a triangle. In another embodiment, the composition filling the one or more than one sample zone is in a solid state. In another embodiment, the one or more than one absorbent comprises a polymeric material in either fibrous or particulate form. In another embodiment, the one or more than one absorbent comprises a hydrophilic material. In another embodiment, the absorbent consists of a single material. In another embodiment, the absorbent comprises a plurality of materials. In another embodiment, the one or more than one absorbent is selected from the group consisting of carbon, cellulose acetate, cellulose beads, cellulose fibers, cellulose particles, dextran fibers, dextran particles, diatomaceous earth, hydroxyapatite, nitrocellulose, nylon, polyesters, polyethylene and silica. In another embodiment, the one or more than one stabilizer comprises a substance selected from the group consisting of a dodecyl sulfate as its sodium, a lithium salt, an anionic salt, a potassium salt, cetyl pyridinium hydrochloride, guinidinium hydrochloride, guinidinium thiocyanate, lithium sulphate and potassium sulphate. In another embodiment, the stabilizer comprises a buffer selected from the group consisting of MOPS and TRIS. In another embodiment, the stabilizer comprises an antioxidant selected from the group consisting of ascorbic acid, disodium ethylene tetra acetic acid (Na₂ EDTA), dithiothreitol, ethyl parabens and methyl parabens. In another embodiment, the stabilizer comprises a substance that inhibits nucleases, such as for example ribonucleases, where the substance is selected from the group consisting of aurine tricarboxylic acid, one or more than one guinidinium salts, placental ribonuclease inhibitor and vanadyl complexes. In another embodiment, the sample zones further comprise a depression in the surface of each sample zone as viewed from the top surface. In another embodiment, the device further comprises a handle. In another embodiment, the handle is a loop.

According to another embodiment of the present invention, there is provided a method of making a device for collecting and preserving nucleic acids in a sample according to the present invention. In one embodiment, the method comprises: a) providing the support; b) providing the one or more than one absorbent, and the one or more than one stabilizer; and c) filling the one or more than one sample zone in the support with the one or more than one absorbent and the one or more than one stabilizer by: i) filling the one or more than one sample zone in the support with the one or more than one absorbent, and then applying the one or more than one stabilizer to the absorbent in each of the sample zones; or ii) producing a composition comprising the one or more than one absorbent and the one or more than one stabilizer, and then filling the one or more than one sample zone in the support with the composition. In one embodiment, the composition is produced by combining the one or more than one absorbent and the one or more than one stabilizer in an aqueous solution to produce a paste or slurry. In another embodiment, the method further comprises removing contaminating nucleic acids from the one or more than one absorbent and one or more than one stabilizer. In another embodiment, the method further comprises treating the absorbent with a wetting agent. In another embodiment, the method further comprises removing any excess absorbent, or excess composition on the device but not in a sample zone. In another embodiment, the method further comprises further comprising drying the absorbent or the composition in the one or more than one sample zone.

According to another embodiment of the present invention, there is provided a method for collecting and preserving nucleic acids in a sample. The method comprises: a) providing a device according to the present invention; b) providing a sample potentially comprising one or more than one nucleic acid; and c) applying part or all of the sample to one or more than one of the sample zones on the device. In one embodiment, the sample is a biological sample. In another embodiment, where the sample is selected from the group consisting of a cell culture, a cell suspension, biopsy aspirates, bone marrow, cerebrospinal fluid, potable water, plasma, serum, urine and whole blood. In another embodiment, the nucleic acids in the sample are selected from the group consisting DNA and RNA. In another embodiment, the nucleic acids in the sample are selected from the group consisting of mRNA, miRNA and mitochondrial RNA. In another embodiment, the nucleic acids in the sample are selected from the group consisting of genomic DNA and mitochondrial DNA. In another embodiment, the sample provided is from a eukaryote. In another embodiment, the sample provided is from a primate. In another embodiment, the sample provided is from a human. In another embodiment, the method further comprises drying the applied sample. In another embodiment, the device provided comprises depressions in the sample zones, and applying the sample to the one or more than one sample zones comprises applying a predetermined amount of sample based on the volume of the depression. In another embodiment, the method further comprises collecting the sample into a vessel before applying the sample to the one or more than one sample zones. In another embodiment, the method further comprises storing the device for a time between 1 minute and 10 years. In another embodiment, the method further comprises storing the device for a time between 1 day and 1 years. In another embodiment, the method further comprises storing the device for a time between 1 day and 100 days. In another embodiment, the method further comprises sealing the device in a protective container before being stored.

According to another embodiment of the present invention, there is provided a method of detecting and quantifying nucleic acids in a sample. The method comprises: a) collecting and preserving nucleic acids in the sample according to the method of the present invention; b) removing the absorbent with sample from the sample zones of the device; and c) detecting, or detecting and quantifying the nucleic acids. In one embodiment, the method further comprises performing a technique selected from the group consisting of PCR, RT-PCR, and quantitative RT-PCR.

### DRAWINGS

These and other features, aspects and advantages of the present invention will become better understood with regard to the following description, appended claims, and accompanying figures where:

FIG. 1 is a top perspective schematic view of a device according to the present invention;

FIG. 2 is a cross-sectional, lateral perspective schematic view of the device according to the present invention as shown in FIG. 1 taken along line 2-2;

FIG. 3 is a bar chart showing the qRT-PCR results of various housekeeping genes at various intervals over a 117-day period using the present method compared to control;

FIG. 4 is a graph showing real-time PCR standard curves of the 18S gene using 10-fold serial dilutions of commercially available cDNA; and

FIG. 5 is a graph showing 18S gene results for test zones obtained day 1 through day 117.

### DESCRIPTION

According to one embodiment of the present invention, there is provided a device for collecting and preserving nucleic acids in a sample. The device comprises a support and at least one sample zone on the support for loading the stabilizer onto the device. In a preferred embodiment, the nucleic acids in the stabilizer are mRNA. According to another embodiment of the present invention, there is provided a method of making a device for collecting and preserving nucleic acids in a sample. In one embodiment, the method comprises providing an absorbent and then filling one or more sample zone in a support with the absorbent, and then applying one or more than one stabilizer to the absorbent. In another embodiment, the method comprises forming a composition by combining one or more than one absorbent and one or more than one stabilizer, and then filling one or more sample zone in a support with the composition. According to another embodiment of the present invention, there is provided a method for collecting and preserving nucleic acids in a sample. In one embodiment, the sample is a biological sample. In one embodiment, the method comprises providing a device for collecting and preserving nucleic acids in a sample according to the present invention. In a preferred embodiment, the nucleic acids in the biological sample are mRNA. According to another embodiment of the present invention, there is provided a method of detecting and quantifying nucleic acids in a sample. In one embodiment, the sample is a biological sample. In another embodiment, the method comprises collecting and preserving nucleic acids in a sample according to a method of the present invention. In a preferred embodiment, the nucleic acids in the sample are mRNA. The device and methods will now be disclosed in greater detail.

As used herein, except where the context requires otherwise, the term “comprise” and variations of the term, such as “comprising”, “comprises” and “comprised” are not intended to exclude other additives, components, integers or steps.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

As used in this disclosure, except where the context requires otherwise, the method steps disclosed and shown are not intended to be limiting nor are they intended to indicate that each step is essential to the method or that each step must occur in the order disclosed.

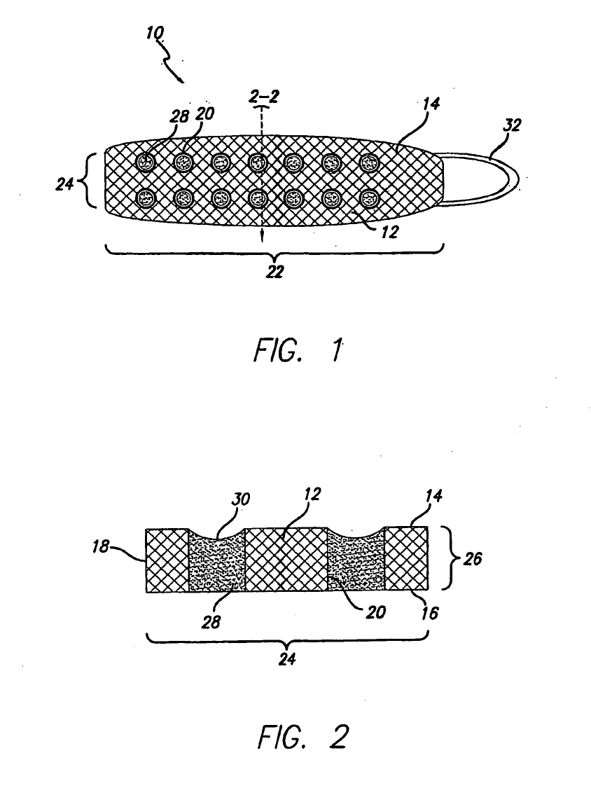

According to one embodiment of the present invention, there is provided a device for collecting and preserving nucleic acids in a sample. In one embodiment, the sample is a biological sample. Referring now to FIG. 1 and FIG. 2, there are shown, respectively, a top perspective schematic view of a device according to the present invention (FIG. 1); and a cross-sectional, lateral perspective schematic view of the device according to the present invention as shown in FIG. 1 taken along line 2-2 (FIG. 2). As can be seen, the device 10 comprises a support 12 comprising a top surface 14, an opposing bottom surface 16, and a lateral edge 18 surrounding the top surface 14 and the bottom surface 16. The support 12 further comprises one or more than one sample zone 20 in the support 12 for loading the sample onto the device 10.

The shape of the device 10 is determined according to the intended use of the device 10, as will be understood by those with skill in the art with reference to this disclosure. In one embodiment, for example, the top surface 14 of the device 10 comprises a shape selected from the group consisting of an oval, a circle, a rectangle, a rectangle with rounded corners, a square, a square with rounded corners, a triangle and a triangle with rounded corners. In a preferred embodiment, the shape of the top surface 14 of the device 10 is a rectangle comprising a long side 22 and a short side 24 as shown in FIG. 1.

The dimensions of the device 10 are also selected according to the intended use of the device 10, as will be understood by those with skill in the art with reference to this disclosure. In a preferred embodiment, the dimensions of the device 10 are selected so that the device 10 is easily portable by hand carrying and easily storable in typical drawers or cabinets in a research lab. For example, in one embodiment, the device 10 is a rectangle with rounded corners comprising a long side 22 and a short side 24 as shown in FIG. 1, and the long side 22 is between 4 cm and 50 cm. In another embodiment, the device 10 is a rectangle with rounded corners comprising a long side 22 and a short side 24 as shown in FIG. 1, and the long side 22 is between 4 cm and 25 cm. In another embodiment, the device 10 is a rectangle with rounded corners comprising a long side 22 and a short side 24 as shown in FIG. 1, and the long side 22 is between 4 cm and 10 cm. In another embodiment, the device 10 is a rectangle with rounded corners comprising a long side 22 and a short side 24 as shown in FIG. 1, and the short side 24 is between 1 cm and 10 cm. In another embodiment, the device 10 is a rectangle with rounded corners comprising a long side 22 and a short side 24 as shown in FIG. 1, and the short side 24 is between 1 cm and 5 cm. In another embodiment, the device 10 is a rectangle with rounded corners comprising a long side 22 and a short side 24 as shown in FIG. 1, and the short side 24 is between 1 cm and 2 cm. In another embodiment, the device 10 is a rectangle with rounded corners comprising a thickness 26 as shown in FIG. 2, and the thickness 26 is between 1 mm and 10 mm. In another embodiment, the device 10 is a rectangle with rounded corners comprising a thickness 26 as shown in FIG. 2, and the thickness 26 is between 1 mm and 5 mm. In another embodiment, the device 10 is a rectangle with rounded corners comprising a thickness 26 as shown in FIG. 2, and the thickness 26 is between 1 mm and 2 mm.

In one embodiment, the support 12 of the device 10 comprises a material that is inert with respect to nucleic acids. In a preferred embodiment, the support 12 comprises a hydrophobic material. In a preferred embodiment, the support 12 comprises a material selected from the group consisting of plasticized cardboard, polyacetate, polycarbonate and polypropylene.

As disclosed above, the support 12 further comprises one or more than one sample zone 20 in the support 12 for loading the sample onto the device 10. In one embodiment, the one or more than one sample zone 20 on the support 12 comprises a recess or space within the support 12 extending from the top surface 14 toward, but not through, the bottom surface 16. In a particularly preferred embodiment, the one or more than one sample zone 20 on the support 12 comprises a space within the support 12 extending from the top surface 14 completely through the bottom surface 16.

In one embodiment, the one or more than one sample zone 20 comprises a plurality of sample zones 20 comprising between 2 and 1000 sample zones 20. In another embodiment, the one or more than one sample zone 20 comprises a plurality of sample zones 20 comprising between 2 and 500 sample zones 20. In another embodiment, the one or more than one sample zone 20 comprises a plurality of sample zones 20 comprising between 20 and 200 sample zones 20.

In one embodiment, the device comprises a plurality of sample zones 20, and the shape of each sample zone 20 is identical to every other sample zone 20. In one embodiment, the device comprises a plurality of sample zones 20, and the shape of at least one sample zone 20 is different that the shape of at least one other sample zone 20. In one embodiment, the shape of at least one of the one or more than one sample zone 20, as viewed from the top surface 14, comprises a shape selected from the group consisting of an oval, a circle, a rectangle, a square and a triangle.

The dimensions of the one or more than one sample zone 20 depend on the dimensions of the support 12, as will be understood by those with skill in the art with reference to this disclosure. By way of example only, in one embodiment, the one or more than one sample zone 20 comprises a recess or space within the support 12 extending from the top surface 14 toward, but not through, the bottom surface 16 maximally between 20% and 90% of the thickness 26 of the support 12. In another embodiment, the one or more than one sample zone 20 comprises a recess or space within the support 12 extending from the top surface 14 toward, but not through, the bottom surface 16 maximally between 30% and 80% of the thickness 26 of the support 12. In another embodiment, the one or more than one sample zone 20 comprises a recess or space within the support 12 extending from the top surface 14 toward, but not through, the bottom surface 16 maximally between 25% and 75% of the thickness 26 of the support 12. By way of example only, in one embodiment, the one or more than one sample zone 20 comprises a recess or space within the support 12 extending from the top surface 14 toward, but not through, the bottom surface 16 maximally between 20% and 90% of the thickness 26 of the support 12.

By way of example only, in one embodiment, each sample zone 20 is round as viewed form the top surface 14, and each sample zone 20 has a diameter between 1 mm and 20 mm. In another embodiment, each sample zone 20 is round as viewed form the top surface 14, and each sample zone 20 has a diameter between 1 mm and 10 mm. In another embodiment, each sample zone 20 is round as viewed form the top surface 14, and each sample zone 20 has a diameter between 1 mm and 5 mm. Further by way of example only, in one embodiment, each sample zone 20 extends from the top surface 14 toward, but not through, the bottom surface 16 maximally between 0.5 mm and 10 mm. In another embodiment, each sample zone 20 extends from the top surface 14 toward, but not through, the bottom surface 16 maximally between 1 mm and 5 mm. In another embodiment, each sample zone 20 extends from the top surface 14 toward, but not through, the bottom surface 16 maximally between 1 mm and 2 mm.

The one or more than one sample zone 20 further comprises a composition 28 comprising a) one or more than one absorbent, and b) one or more than one stabilizer. In a preferred embodiment, the composition 28 filling the one or more than one sample zone 20 is in a solid state.

The one or more than one absorbent functions to absorb the sample onto the sample zone 20 without irreversibly binding the nucleic acids in the sample. The absorbent comprises a material that can be dried after application to the sample zone 20 without powdering or otherwise breaking or detaching from the surface of the sample zone 20, without the application of an external force to cause detachment of the composition from the walls of the sample zone 20.

In one embodiment, the one or more than one absorbent comprises a polymeric material in either fibrous or particulate form. In another embodiment, the one or more than one absorbent comprises a hydrophilic material. In one embodiment, the absorbent consists of a single material. In another embodiment, the absorbent comprises a plurality of materials. In another embodiment, the one or more than one absorbent is selected from the group consisting of carbon, cellulose acetate, cellulose beads, cellulose fibers, cellulose particles, dextran fibers, dextran particles, diatomatious earth, hydroxyapatite, nitrocellulose, nylon, polyesters, polyethylene and silica. In a preferred embodiment, the absorbent comprises a material which is substantially free (that is more than 99.9%) of heavy metals or other constituents which cause nucleic acids to breakdown. In another preferred embodiment, the absorbent comprises a material which is substantially free (that is more than 99.9%) of nucleic acids which can arise from the use of materials produced from fermentation.

The one or more than one stabilizer functions to diminish or prevent breakdown of the nucleic acid in the sample, such as for example, by denaturing proteins that can inactivate polynucleotides, that can sequester polynucleotides, and by facilitating the disassociation of nucleases from nucleic acids, thereby freeing the nucleic acids form interference by such nucleases.

In a preferred embodiment, the one or more than one stabilizer performs one or more than one function selected from the group consisting of antioxidation, buffering, cell lysis, chelation of metal cofactors such as calcium or magnesium, nuclease inhibition, and protection of nucleic acids from oxidative degradation or the action of microbial contaminants. When used as a buffer, the stabilizing should maintain pH between 6.0 and 8.0, preferably between pH 6.2 and 7.0, and most preferably between 6.4 and 6.8. In another embodiment, the one or more than one stabilizer comprises a substance selected from the group consisting of a dodecyl sulfate as its sodium, a lithium salt, an anionic salt, a potassium salt, cetyl pyridinium hydrochloride, guanidinium hydrochloride, guanidinium thiocyanate, lithium sulphate and potassium sulphate. In one embodiment, the stabilizer comprises a buffer selected from the group consisting of MOPS and TRIS. In another embodiment, the stabilizer comprises an antioxidant that decreases or prevents oxidative degradation of the nucleic acids selected from the group consisting of ascorbic acid, disodium ethylene tetra acetic acid (Na₂ EDTA), dithiothreitol, ethyl parabens and methyl parabens. In another embodiment, the stabilizer comprises a substance that inhibits nucleases, such as for example ribonucleases, where the substance is selected from the group consisting of aurine tricarboxylic acid, one or more than one guanidinium salts, placental ribonuclease inhibitor and vanadyl complexes. In one embodiment, the amount of the one or more than one stabilizers is sufficient to preserve and stabilize the nucleic acids in the sample, as will be understood by those with skill in the art with reference to this disclosure.

As will be understood by those with skill in the art with reference to this disclosure, the absolute and relative amounts of the absorbent and stabilizer present in each sample zone 20 is selected to be sufficient for the purposes disclosed in this disclosure.

In one embodiment, the composition further comprises an additive that functions to bind the absorbent into a solid phase or to the walls of the sample zone 20, or both to bind the absorbent into a solid phase and to the walls of the sample zone 20. In one embodiment, the additive is selected from the group consisting of albumin, gelatin, polyvinyl alcohol, starch, sucrose, trehalose, polyacrylamide, and polyethylene glycol.

In one embodiment, the sample zones 20 further comprise a depression 30 in the surface of each sample zone 20 as viewed from the top surface 14. The depression 30 is configured to accept a predetermined sample volume. By way of example only, in one embodiment, the depression 30 comprises a volume, as measured from the plane of the top surface 14 to the surface of the composition within the sample zone 20 of between 1 and 100 μl. In another embodiment, the depression 30 comprises a volume, as measured from the plane of the top surface 14 to the surface of the composition within the sample zone 20 of between 2 and 50 μl. In another embodiment, the depression 30 comprises a volume, as measured from the plane of the top surface 14 to the surface of the composition within the sample zone 20 of between 5 and 20 μl.

In one embodiment, the device 10 further comprises a handle 32 to assist in manually grasping and manipulating the device. The handle 32 can be any suitable size and shape for the intended purpose as will be understood by those with skill in the art with reference to this disclosure. In one embodiment, the handle 32 comprises a loop as shown to permit storage of a plurality of devices 10 by threading the open portion of the loop onto a strut or peg.

According to another embodiment of the present invention, there is provided a method of making a device for collecting and preserving nucleic acids in a sample. In one embodiment, the device made according to this method is useful for collecting and preserving mRNA in the stabilizer. In a preferred embodiment, the device made according to this method is a device 10 according to the present invention.

In one embodiment, the method comprises, first, providing a support, such as a support 12 comprising one or more than one sample zone 20, as disclosed in connection with the device 10 according to the present invention. In a preferred embodiment, the support is produced using injection molding to form a plastic material into the desired shape, however, any suitable technique can be used, as will be understood by those with skill in the art with reference to this disclosure. In one embodiment, the method comprises forming one or more than one sample zone in the support by drilling or by an equivalent technique, as will be understood by those with skill in the art with reference to this disclosure.

In a preferred embodiment, the shape and dimensions of the support are in accordance with the shape and dimensions disclosed in connection with the support 12 for the device 10.

Next, the method comprises providing one or more than one absorbent, and one or more than one stabilizer in accordance with the absorbents and stabilizers as disclosed in connection with the device 10. In one embodiment, the method comprises filling the one or more than one sample zone in the support with the one or more than one absorbent, and then applying the one or more than one stabilizer to the absorbent in each of the sample zones. In an alternate embodiment, the method comprises producing a composition comprising the one or more than one absorbent and the one or more than one stabilizer, and then filling the one or more than one sample zone in the support with the composition. In a preferred embodiment, the composition provided is a composition 28, as disclosed in connection with the device 10 according to the present invention, comprising a) one or more than one absorbent, and b) one or more than one stabilizer. In one embodiment, the composition is produced by combining the one or more than one absorbent and the one or more than one stabilizer in an aqueous solution to produce a paste or slurry.

By way of example, an absorbent was produced from 20 micron cellulose Sigmacell Type 101 (Sigma, St. Louis, Mo. US) particles suspended in water and formed into a paste. The paste was then applied to each of 10 sample zones comprising round through holes present in a rectangular polymer support, and the support with the absorbent filled sample zones was allowed to air dry at 50° C. for 2 hours. A stabilizer comprising an aqueous solution of 1% Sodium Dodecyl Sulfate, 10 mM EDTA, 10 mM MOPS, 500 mM lithium chloride and 5 mM ammonium salt of aurine tricarboxylic acid, pH 6.8 (all from Sigma) was prepared, and the stabilizer was applied to the absorbent in each of five of the ten sample zones. The device was then allowed to dry for 2 hours at 50° C.

In another embodiment, the method comprises placing less of the absorbent, or less of the composition, in each of the one or more than one of the sample zones than is needed to fill the sample zone, thereby leaving a depression, as disclosed in connection with the device 10.

In a preferred embodiment, the method further comprises removing contaminating nucleic acids from the one or more than one absorbent and one or more than one stabilizer using techniques as will be understood by those with skill in the art with reference to this disclosure, such as for example, the addition of one or more than one nuclease to the one or more than one absorbent and one or more than one stabilizer, allowing the one or more than one nuclease to degrade any contaminating nucleic acid, and then removing, inactivating, or both removing and inactivating, the one or more than one nuclease following their use to degrade any contaminating nucleic acids.

In another embodiment, the method further comprises treating the absorbent with a wetting agent to render a substantially hydrophobic material, such as nylon, suitable for use in the composition. In one embodiment, the wetting agent is selected from the group consisting of a surfactant, an ionic detergent and a nonionic detergent.

In a preferred embodiment, the method further comprises removing any excess absorbent, or excess composition on the device but not in a sample zone, such as for example by wiping the excess absorbent or excess composition from the top surface and the bottom surface of the device.

In one embodiment, the method further comprises drying the absorbent or the composition in the one or more than one sample zone. Drying can be accomplished passively by air drying at room temperature, or can be accomplished by the application of heat, the application of a vacuum, or the application of both heat and a vacuum, as will be understood by those with skill in the art with reference to this disclosure. Drying functions to remove bulk water or other solvents from the absorbent or from the composition.

In another embodiment, the method comprises filling the one or more than one sample zone in the support with the one or more than one absorbent, and then applying the one or more than one stabilizer to the absorbent in each of the sample zones, and the method further comprises drying the absorbent with the applied stabilizer. Drying can be accomplished passively by air drying at room temperature, or can be accomplished by the application of heat, the application of a vacuum, or the application of both heat and a vacuum, as will be understood by those with skill in the art with reference to this disclosure. Drying functions to remove bulk water or other solvents from the absorbent or from the composition.

According to another embodiment of the present invention, there is provided a method for collecting and preserving nucleic acids in a sample. In one embodiment, the method comprises providing a device for collecting and preserving nucleic acids in a sample according to the present invention. In a preferred embodiment, the device is a device 10 according to the present invention.

Next, the method comprises providing a sample potentially comprising one or more than one nucleic acid. In one embodiment, the sample is a biological sample. In a preferred embodiment, the sample is selected from the group consisting of a cell culture, a cell suspension, biopsy aspirates, bone marrow, cerebrospinal fluid, potable water, plasma, serum, urine and whole blood. In another embodiment, the nucleic acids in the sample are selected from the group consisting DNA and RNA. In a preferred embodiment, the nucleic acids in the sample are selected from the group consisting of mRNA, miRNA and mitochondrial RNA. In a preferred embodiment, the nucleic acids in the sample are selected from the group consisting of genomic DNA and mitochondrial DNA.

In one embodiment, the sample provided is from a eukaryote. In another embodiment, the sample provided is from a primate. In another embodiment, the sample provided is from a human. In a preferred embodiment, providing the sample comprises obtaining whole blood from a human by a finger stick.

Next, part or all of the sample is applied to one or more than one of the sample zones on the device. In a preferred embodiment, the applied sample is then allowed to dry while protected from contamination, under conditions suitable for the nucleic acid being collected and preserved, as will be understood by those with skill in the art with reference to this disclosure. For example, drying can be accomplished passively by air drying at room temperature, or can be accomplished by the application of heat, the application of a vacuum, or the application of both heat and a vacuum, as will be understood by those with skill in the art with reference to this disclosure. Drying functions to remove bull water or other solvents from the absorbent or from the composition.

In another embodiment, between 1 μl and 1000 μl of sample is applied to each sample zone. In another embodiment, between 1 μl and 100 μl of sample is applied to each sample zone. In another embodiment, between 1 μl and 10 μl of sample is applied to each sample zone.

In a preferred embodiment, the device provided comprises depressions in the sample zones, and applying the sample to the one or more than one sample zones comprises applying a predetermined amount of sample based on the volume of the depression.

In one embodiment, the method further comprises collecting the sample into a vessel before applying the sample to the one or more than one sample zones. Collecting the sample into a vessel before applying the sample to the sample zone assists in assuring that a predetermined amount of the sample is applied, as will be understood by those with skill in the art with reference to this disclosure. In one embodiment, the vessel is selected from the group consisting of a capillary tube and a pipette tip collection tube.

Once, the sample zones are dry, the device with the nucleic acids in the sample zones can be stored for future use. In one embodiment, the device is stored for a time between 1 minute and 10 years. In another embodiment, the device is stored for a time between 1 day and 1 year. In another embodiment, the device is stored for a time between 1 day and 100 days.

In another embodiment, the device is sealed in a protective container before being stored. In one embodiment, the protective container is selected from the group consisting of a tube and a box.

In one embodiment, the device is stored at ambient temperature. In another embodiment, the device is stored at a temperature between −20° C. and −80° C. In another embodiment, the device is stored at a temperature between 0° C. and −100° C.

According to another embodiment of the present invention, there is provided a method of detecting and quantifying nucleic acids in a sample. In one embodiment, the method comprises, first, collecting and preserving nucleic acids in a sample according to a method of the present invention.

In one embodiment, the sample is a biological sample. In a preferred embodiment, the sample is selected from the group consisting of a cell culture, a cell suspension, biopsy aspirates, bone marrow, cerebrospinal fluid, potable water, plasma, serum, urine and whole blood. In another embodiment, the nucleic acids in the sample are selected from the group consisting DNA and RNA. In a preferred embodiment, the nucleic acids in the sample are selected from the group consisting of mRNA, miRNA and mitochondrial RNA. In a preferred embodiment, the nucleic acids in the sample are selected from the group consisting of genomic DNA and mitochondrial DNA.

Next, the method comprises removing the absorbent with sample from the sample zones of the device. In one embodiment, removing comprises pushing the absorbent with the sample through the sample zone, thereby detaching the absorbent with the sample from the device. In another embodiment, removing comprises scooping the absorbent with the sample from the sample zone, thereby detaching the absorbent with the sample from the device.

Then, the absorbent with the sample is processed by methods well known in the art to obtain isolated nucleic acids such as DNA or RNA, including mRNA and micro RNAs, to remove the nucleic acids from the absorbent.

The nucleic acids are then subject to detection and quantification using standard techniques, as will be understood by those with skill in the art with reference to this disclosure, such as for example, amplification methods selected from the group consisting of PCR, RT-PCR, and quantitative RT-PCR.

In one embodiment, the method comprises eluting the nucleic acids from the absorbent using standard techniques, as will be understood by those with skill in the art with reference to this disclosure. Next, the nucleic acid is isolated from the eluted material using standard techniques, as will be understood by those with skill in the art with reference to this disclosure.

By way of example, the above steps were performed as follows. After application of 10 μl of whole blood to each sample zone, the blood was allowed to absorb and to visibly dry. Control and test blood zones were then punched from the device into individual tubes using a small plastic dowel for each of the successive time points. The punches for the first three time points were stored at −80° C. until they could be processed. The punches were each processed by adding 100 μl of the above lysis preservation buffer to each, and completely dispersing the cellulose punches containing the applied blood into this solution. Then, 5 μl of poly-dT paramagnetic beads (Dynabeads, Dynal Biotech, L.L.C., Brown Deer, Wis. US) was added to the suspension of dispersed blood spots or punches and was allowed to incubate at room temperature for 4 minutes with repeated mixing by inversion. Paramagnetic beads were separated from the mixture components by inversion of the capped tubes containing the mixture and application of a magnet to the cap of the inverted tube for about 2 minutes. Next, the tubes were placed upright, allowing the liquid phase to drain into the tube while the magnet applied to the cap retained the mRNAs captured by the poly-dT paramagnetic beads. Each cap was then transferred to a fresh tube containing Wash Buffer A (Dynal Dynal Biotech). Then, the magnet was withdrawn from the cap, and the beads and wash buffer were mixed and incubated for about 2 minutes at room temperature. The cap transfer and incubation steps were repeated as described above using 2 fresh tubes of 200 μl each of Wash Buffer B (Dynal Dynal Biotech). The mRNAs were eluted from the beads by the addition of 10 μl 10 mM Tris-HCl pH 7.5 (Dynal Dynal Biotech) and heating to 60° C. for 5 minutes followed by centrifugation at 5000×g to pellet the beads. The supernatant containing the mRNA was transferred to a fresh tube and stored at −80° C. until processing to produce cDNA and qRT-PCR by standard methods known in the art.

In one embodiment, the method further comprises quantifying the isolated nucleic acids using standard techniques, as will be understood by those with skill in the art with reference to this disclosure. In another embodiment, the method further comprises performing expression analysis on the isolated nucleic acids using standard techniques, as will be understood by those with skill in the art with reference to this disclosure. In a preferred embodiment, the expression analysis is qRT-PCR.

By way of example, the method for the isolation of mRNA, and for downstream analysis from finger-stick whole blood on an absorbent matrix according to the present invention was performed as follows. First, 10 μl whole blood was collected using a finger stick and collection of the whole blood into a pipette tip collection or capillary tube. Next, the whole blood was placed on multiple absorbent matrix zones containing lysing and stabilizing agents to prevent RNA degradation.

Then, mRNA isolation was completed for each zone by eluting the sample from the absorbent matrix, isolation of mRNA using oligo(dT) magnetic bead techniques and elution of mRNA in a stabilizing buffer. Next, the isolated mRNA was quantified using a Nanodrop® spectrophotometer (NanoDrop Technologies, Inc., Wilmington, Del. US).

Then, expression analysis was performed on the isolated mRNA by qRT-PCR using TaqMan® gene expression assays (Applied Biosystems; Foster City, Calif. US) for a set of high, medium and low expression housekeeping genes (18S, GAPDH, GUSb, PGK, TBP). Next, the mRNA was isolated from sample replicate matrix zones at successive intervals extending to 117 days.

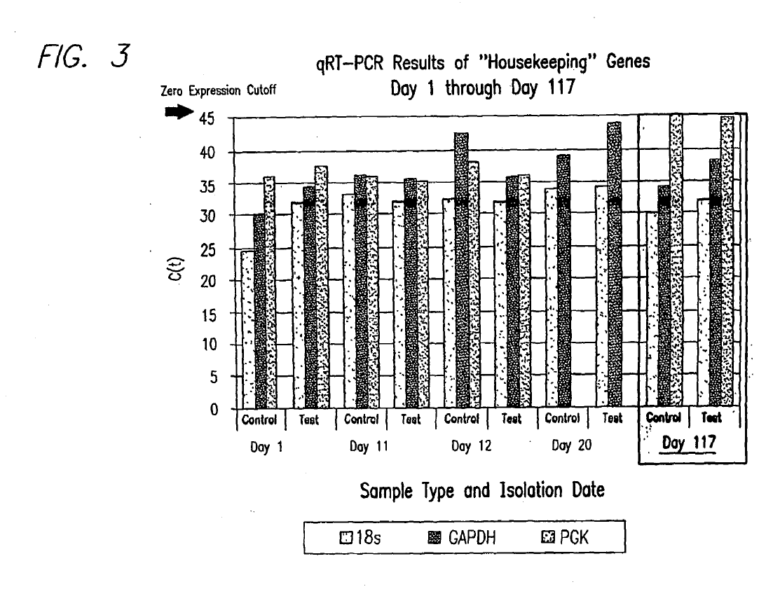

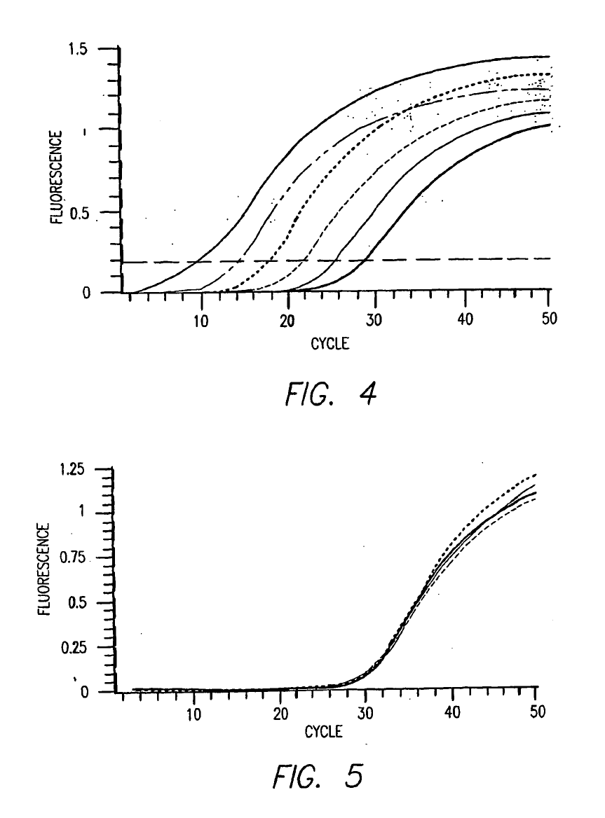

Referring now to FIG. 3, FIG. 4 and FIG. 5, there are shown respectively, a bar chart showing the qRT-PCR results of various housekeeping genes at various intervals over a 117-day period using the present method compared to control (FIG. 3); a graph showing real-time PCR standard curves of the 18S gene using 10-fold serial dilutions of commercially available cDNA (from Princeton BioMeditech Corporation, Princeton, N.J. US). Day 20 showed no detectable expression of the PGK gene and no time point showed detectable expression of GUSb or TBP (FIG. 4); and a graph showing 18S gene results for test zones obtained day 1 through day 117 (FIG. 5). As can be seen, the expression levels determined from the qRT-PCR panel of housekeeping genes remain relatively consistent across time points out to the 117-day limit tested, even when stored at ambient conditions in a sealed container. The results were comparable to those from mRNA obtained from commercially available RNA blood tubes and isolation methods that require using much larger quantities of whole blood and elaborate collection and processing protocols.

Therefore, using the present method, the quality and quantity of mRNA required for qRT-PCR and amplification can successfully be obtained from a finger-stick collection of whole blood on an absorbent matrix. This is particularly useful in circumstances in which peripheral blood is suitable for expression analysis but where full scale collections are logistically difficult and or costly.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

1. A device for collecting and preserving nucleic acids in a sample, the device comprising:
a) a support comprising a top surface, an opposing bottom surface, and a lateral edge surrounding the top surface and the bottom surface;
b) one or more than one sample zone in the support for loading the sample onto the device; and
c) a composition comprising i) one or more than one absorbent, and ii) one or more than one stabilizer in a solid state;
where the one or more than one sample zone on the support comprises a recess or space within the support extending from the top surface toward, but not through, the bottom surface, or comprises a space within the support extending from the top surface completely through the bottom surface; and
where the composition is retained inside the recess or space of the sample zone.

2. The device of claim 1, where the support comprises a hydrophobic material.

3. The device of claim 1, where the support comprises a material selected from the group consisting of plasticized cardboard, polyacetate, polycarbonate and polypropylene.

4. The device of claim 1, further comprising a shape selected from the group consisting of an oval, a circle, a rectangle, a rectangle with rounded corners, a square, a square with rounded corners, a triangle and a triangle with rounded corners.

5. The device of claim 1, where the one or more than one sample zone comprises a plurality of sample zones comprising between 2 and 1000 sample zones.

6. The device of claim 1, where the one or more than one sample zone comprises a plurality of sample zones comprising between 2 and 500 sample zones.

7. The device of claim 1, where the one or more than one sample zone comprises a plurality of sample zones comprising between 20 and 200 sample zones.

8. The device of claim 1, where the device comprises a plurality of sample zones, and the shape of each sample zone is identical to every other sample zone.

9. The device of claim 1, where the device comprises a plurality of sample zones, and the shape of at least one sample zone is different that the shape of at least one other sample zone.

10. The device of claim 1, where the shape of at least one of the one or more than one sample zone, as viewed from the top surface, comprises a shape selected from the group consisting of an oval, a circle, a rectangle, a square and a triangle.

11. The device of claim 1, where the composition filling the one or more than one sample zone is in a solid state.

12. The device of claim 1, where the one or more than one absorbent comprises a polymeric material in either fibrous or particulate form.

13. The device of claim 1, where the one or more than one absorbent comprises a hydrophilic material.

14. The device of claim 1, where the absorbent consists of a single material.

15. The device of claim 1, where the absorbent comprises a plurality of materials.

16. The device of claim 1, where the one or more than one absorbent is selected from the group consisting of carbon, cellulose acetate, cellulose beads, cellulose fibers, cellulose particles, dextran fibers, dextran particles, diatomatious earth, hydroxyapatite, nitrocellulose, nylon, polyesters, polyethylene and silica.

17. The device of claim 1, where the one or more than one stabilizer comprises a substance selected from the group consisting of a dodecyl sulfate as its sodium, a lithium salt, an anionic salt, a potassium salt, cetyl pyridinium hydrochloride, guinidinium hydrochloride, guinidinium thiocyanate, lithium sulphate and potassium sulphate.

18. The device of claim 1, where the stabilizer comprises a buffer selected from the group consisting of MOPS and TRIS.

19. The device of claim 1, where the stabilizer comprises an antioxidant selected from the group consisting of ascorbic acid, disodium ethylene tetra acetic acid (Na₂ EDTA), dithiothreitol, ethyl parabens and methyl parabens.

20. The device of claim 1, where the stabilizer comprises a substance that inhibits nucleases, such as for example ribonucleases, where the substance is selected from the group consisting of aurine tricarboxylic acid, one or more than one guinidinium salts, placental ribonuclease inhibitor and vanadyl complexes.

21. The device of claim 1, where the composition further comprises an additive selected from the group consisting of albumin, gelatin, polyvinyl alcohol, starch, sucrose, trihalose, polyacrylamide, and polyethylene glycol.

22. The device of claim 1, where the sample zones further comprise a depression in the surface of each sample zone as viewed from the top surface.

23. The device of claim 1, further comprising a handle.

24. The device of claim 23, where the handle is a loop.

25. A method of making a device for collecting and preserving nucleic acids in a sample according to claim 1, the method comprising:
a) providing the support;
b) providing the one or more than one absorbent, and the one or more than one stabilizer; and
c) filling the one or more than one sample zone in the support with the one or more than one absorbent and the one or more than one stabilizer by:
i) filling the one or more than one sample zone in the support with the one or more than one absorbent, and then applying the one or more than one stabilizer to the absorbent in each of the sample zones; or
ii) producing a composition comprising the one or more than one absorbent and the one or more than one stabilizer, and then filling the one or more than one sample zone in the support with the composition.

26. The method of claim 25, where the composition is produced by combining the one or more than one absorbent and the one or more than one stabilizer in an aqueous solution to produce a paste or slurry.

27. The method of claim 25, further comprising removing contaminating nucleic acids from the one or more than one absorbent and one or more than one stabilizer.

28. The method of claim 25, further comprising treating the absorbent with a wetting agent.

29. The method of claim 25, further comprising removing any excess absorbent, or excess composition on the device but not in a sample zone.

30. The method of claim 25, further comprising drying the absorbent or the composition in the one or more than one sample zone.

31. A method for collecting and preserving nucleic acids in a sample, the method comprising:
a) providing a device of claim 1;
b) providing a sample potentially comprising one or more than one nucleic acid; and
c) applying part or all of the sample to one or more than one of the sample zones on the device.

32. The method of claim 31, where the sample is a biological sample.

33. The method of claim 32, where the sample is selected from the group consisting of a cell culture, a cell suspension, biopsy aspirates, bone marrow, cerebrospinal fluid, potable water, plasma, serum, urine and whole blood.

34. The method of claim 31, where the nucleic acids in the sample are selected from the group consisting of DNA and RNA.

35. The method of claim 31, where the nucleic acids in the sample are selected from the group consisting of mRNA, miRNA and mitochondrial RNA.

36. The method of claim 31, where the nucleic acids in the sample are selected from the group consisting of genomic DNA and mitochondrial DNA.

37. The method of claim 31, where the sample provided is from a eukaryote.

38. The method of claim 31, where the sample provided is from a primate.

39. The method of claim 31, where the sample provided is from a human.

40. The method of claim 31, further comprising drying the applied sample.

41. The method of claim 31, where the device provided comprises depressions in the sample zones, and applying the sample to the one or more than one sample zones comprises applying a predetermined amount of sample based on the volume of the depression.

42. The method of claim 31, further comprising collecting the sample into a vessel before applying the sample to the one or more than one sample zones.

43. The method of claim 31, further comprising storing the device for a time between 1 minute and 10 years.

44. The method of claim 31, further comprising storing the device for a time between 1 day and 1 years.

45. The method of claim 31, further comprising storing the device for a time between 1 day and 100 days.

46. The method of claim 31, further comprising sealing the device in a protective container before being stored.

47. The method of detecting and quantifying nucleic acids in a sample, the method comprising:
a) collecting and preserving nucleic acids in the sample according to the method of claim 31;
b) removing the absorbent with sample from the sample zones of the device; and
c) detecting, or detecting and quantifying the nucleic acids.

48. The method of claim 47, where detecting, or detecting and quantifying the nucleic acids comprises performing a technique selected from the group consisting of PCR, RT-PCR, and quantitative RT-PCR.
